(19) United States
(12) Patent Application Publication
(10) Pub. No.: US 2024/0091054 A1
(43) Pub. Date: Mar. 21, 2024

(54) On-Pad Fluid Line Connectors for Arctic Gel Pads

(71) Applicant: C. R. Bard, Inc., Franklin Lakes, NJ (US)

(72) Inventors: Tyghe E. Boone-Worthman, Brighton, CO (US); Madeline Stich, Thornton, CO (US); Karthik Ganesan, Longmont, CO (US); Sean E. Walker, Platteville, CO (US)

(21) Appl. No.: 18/273,163

(22) PCT Filed: Jan. 19, 2022
(86) PCT No.: PCT/US22/13007

§ 371 Date: Jul. 19, 2023

Related U.S. Application Data
(60) Provisional application No. 63/139,673, filed on Jan. 20, 2021.

(51) Int. Cl. A61F 7/02

(52) U.S. Cl. CPC A61F 7/02; A61F 2007/0056

(57) ABSTRACT

Disclosed herein are systems and methods for providing targeted temperature management (TTM) therapy to a patient. For example, TTM systems can include rotatable connector systems for coupling a fluid deliver line to a thermal contact pad. Embodiments of the connector system can include connector pairs that comprise one lumen or multiple lumens. The TTM system includes embodiments where a second thermal pad is coupled to a first thermal pad.

### PRIORITY

This application claims the benefit of priority to U.S. Provisional Application No. 63/139,673, filed Jan. 20, 2021, which is incorporated by reference in its entirety into this application.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g. water) through one or more thermal contact pads coupled to a patient to affect surface-to-surface thermal energy exchange with the patient. In general, TTM systems comprise a TTM fluid control module coupled to at least one contact pad via a fluid deliver line. One such TTM system is disclosed in U.S. Pat. No. 6,645,232, titled “Patient Temperature Control System with Fluid Pressure Maintenance” filed Oct. 11, 2001 and one such thermal contact pad and related system is disclosed in U.S. Pat. No. 6,197,045 titled “Cooling/heating Pad and System” filed Jan. 4, 1999, both of which are incorporated herein by reference in their entireties. As noted in the '045 patent, the ability to establish and maintain intimate pad-to-patient contact is of importance to fully realizing medical efficacies with TTM systems.

As these and other medical applications have evolved, the flexibility of pad placement and the accommodation of different patient sizes have become more important.

Disclosed herein are embodiments of devices and methods for the transportation of a TTM fluid to a thermal contact pad coupled to a patient.

### SUMMARY OF THE INVENTION

Briefly summarized, disclosed herein is a targeted temperature management (TTM) system including a TTM module configured to provide a TTM fluid, a thermal pad configured to receive the TTM fluid from the TTM module to facilitate thermal energy transfer between the TTM fluid and a patient and a fluid delivery line (FDL) extending between the TTM module and the thermal pad, the FDL configured to provide TTM fluid flow between the TTM module and the thermal pad. The thermal pad may comprise a pad connector coupled to a corresponding FDL connector of the FDL, and the FDL connector may be rotatable with respect to the pad connector.

The FDL connector may comprise an elbow to facilitate parallel orientation of a distal portion of the FDL with respect to the pad. The FDL may multi-luminal and the FDL connector may be configured to couple to the pad connector via a snap fit.

At least one of the FDL connector or the pad connector may comprise a sealing member and the other of the FDL connector or the pad connector may comprise an annular sealing surface. The sealing member and the annular sealing surface may be configured to facilitate a fluid seal between the FDL connector and the pad connector.

The system may comprise a filter disposed in line with a TTM fluid flow path of the TTM system. The filter may comprise a porous wall disposed parallel to a flow direction of TTM fluid along the TTM fluid flow path. The filter may be attached to the thermal pad and may further be disposed within a fluid containing layer of the thermal pad.

In some embodiments, the system comprises a second thermal pad and a second FDL extending between the thermal pad and the second thermal pad and the second FDL is configured to provide TTM fluid flow between thermal pad and the second thermal pad. The thermal pad may comprise a second pad connector coupled to a corresponding second FDL connector of the second FDL, and the second FDL connector may be rotatable with respect to the second pad connector. The second thermal pad may also comprise a third pad connector coupled to a corresponding third FDL connector of the second FDL, and the third FDL connector may be rotatable with respect to the third pad connector.

Also disclosed herein is a thermal pad for facilitating thermal energy transfer between a TTM fluid and a patient. The thermal pad may comprise a fluid line configured to transport a TTM fluid between a TTM fluid source and the thermal pad. The thermal pad includes a fluid containing layer configured to contain the TTM fluid and to circulate the TTM fluid within the fluid containing layer to facilitate thermal energy transfer between the TTM fluid and the patient. The fluid line may be coupled to the fluid containing layer such that a distal portion of the fluid line is oriented parallel to the pad and the fluid line may be rotatable with respect the fluid containing layer.

The fluid containing layer of the thermal pad comprises a TTM fluid flow path, and the pad may further include a filter disposed in line with the TTM fluid flow path.

Also disclosed herein is a thermal pad for facilitating thermal energy transfer between a targeted temperature management (TTM) fluid and a patient and the pad comprises a fluid containing layer that is configured to receive a TTM fluid from a TTM fluid source via a fluid line extending between the fluid source and the thermal pad, contain the TTM fluid, and circulate the TTM fluid within the fluid containing layer to facilitate thermal energy transfer between the TTM fluid and the patient. A connector may be attached to the fluid containing layer and the connector may define a fluid port of the fluid containing layer. The connector may be configured to couple to a corresponding fluid line connector to establish fluid communication between the fluid containing layer and the fluid line via a lumen of the connector, and the connector may be configured to swivel with respect to the corresponding fluid line connector.

The connector of the thermal pad may comprise a first lumen for transporting TTM fluid to the fluid containing layer and a second lumen for transporting TTM fluid away from fluid containing layer. The connector may also be configured to couple to the corresponding fluid line connector via a snap fit.

The connector of the thermal pad may comprise an annular sealing surface configured to facilitate a fluid seal between the connector and the corresponding fluid line connector. The connector of the thermal pad may also comprise a second annular sealing surface configured to facilitate a fluid seal between the connector and the corresponding fluid line connector.

The thermal pad may comprise a second connector attached to the fluid containing layer and the second connector may define a second fluid port of the fluid containing layer. The second connector may be configured to couple to a second corresponding fluid line connector of a second fluid line to establish fluid communication between the fluid containing layer and the second fluid line via a lumen of the second connector. The second connector may also be configured to swivel with respect to the second corresponding fluid line connector when the second connector is coupled to the second corresponding fluid line connector.

In some embodiments, the second connector comprises a septum extending across the lumen of the second connector to seal the lumen of the second connector. The septum may be configured to be ruptured when the second connector is coupled to the second corresponding fluid line connector.

The thermal pad may also comprise a third connector attached to the fluid containing layer and the third connector may define a third fluid port of the fluid containing layer. The third connector may be configured to couple to a third corresponding fluid line connector, and the third connector may be configured to swivel with respect to the third corresponding fluid line connector when the third connector is coupled to the third corresponding fluid line connector.

Disclosed herein also is a method of using a targeted temperature management (TTM) system. The method may include providing a TTM system may comprising a TTM module configured to provide a TTM fluid, a thermal pad configured to receive the TTM fluid from the TTM module to facilitate thermal energy transfer between the TTM fluid and a patient, and a fluid delivery line (FDL) extending between the TTM module and the thermal pad, where the FDL is configured to provide TTM fluid flow between the TTM module and the thermal pad. The method further includes connecting the FDL with the pad connector via a rotatable connection, rotating the pad with respect to the FDL from a first orientation of the thermal pad to a second orientation of the thermal pad, applying the pad to the patient in the second orientation, and delivering TTM fluid from the TTM module to the thermal pad.

The TTM system may further comprise a second thermal pad and a second FDL, and the method may further comprise coupling the second FDL to the thermal pad via a rotatable connection and coupling the second FDL to the second thermal pad via a rotatable connection. The method may further include rotating the second FDL with respect to the thermal pad from a first orientation of the second FDL to a second orientation of the second FDL. The method may further include rotating the second thermal pad with respect to the second FDL from a first orientation of the second thermal pad to a second orientation of the second thermal pad, applying the second pad to the patient, and delivering TTM fluid from the thermal pad to the second thermal pad.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a patient and a targeted temperature management (TTM) system for cooling or warming the patient, in accordance with some embodiments.

FIG. 2 illustrates a hydraulic schematic of the TTM system of FIG. 1, in accordance with some embodiments.

FIG. 3 illustrates a block diagram depicting various elements of a console of the TTM module of FIG. 1, in accordance with some embodiments.

FIG. 4A is a top view of a portion of the thermal contact pad of FIG. 1, in accordance with some embodiments.

FIG. 4B is a cross-sectional side view of the portion the thermal contact pad of FIG. 4A, in accordance with some embodiments.

FIG. 5A is a cross-sectional side view of a conduit set of fluid delivery line of FIG. 1, in accordance with some embodiments.

FIGS. 5B-5F provide various cross-sectional end views of the conduit set depicted in FIG. 5A, in accordance with some embodiments.

FIG. 6A is a cross-sectional side view of a connector set in a non-connected state, in accordance with some embodiments.

FIG. 6B is a cross-sectional side view of the connector set of FIG. 6A in a connected state, in accordance with some embodiments.

FIG. 7A is a cross-sectional side view of a multi-luminal connector set in a non-connected state, in accordance with some embodiments.

FIG. 7B is a cross-sectional side view of the multi-luminal connector set of FIG. 7A in a connected state, in accordance with some embodiments.

FIG. 7C is a cross-sectional side view of a cap of the connector set of FIG. 7A, in accordance with some embodiments.

FIG. 8A provides an exploded perspective view of a TTM fluid filter, in accordance with some embodiments.

FIG. 8B provides a cross-sectional side view of the filter of FIG. 8A, in accordance with some embodiments.

FIG. 8C provides a side cross-sectional view of the thermal contact pad of FIG. 1 incorporating the filter of FIG. 8A, in accordance with some embodiments.

FIG. 9A illustrates a targeted temperature management (TTM) system including a first thermal pad and a second thermal pad, in accordance with some embodiments.

FIG. 9B illustrates an exploded assembly view of the first thermal pad and the second thermal pad of FIG. 9A, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Unless defined otherwise, all technical and scientific terms used herein have the same meaning as commonly understood by those of ordinary skill in the art.

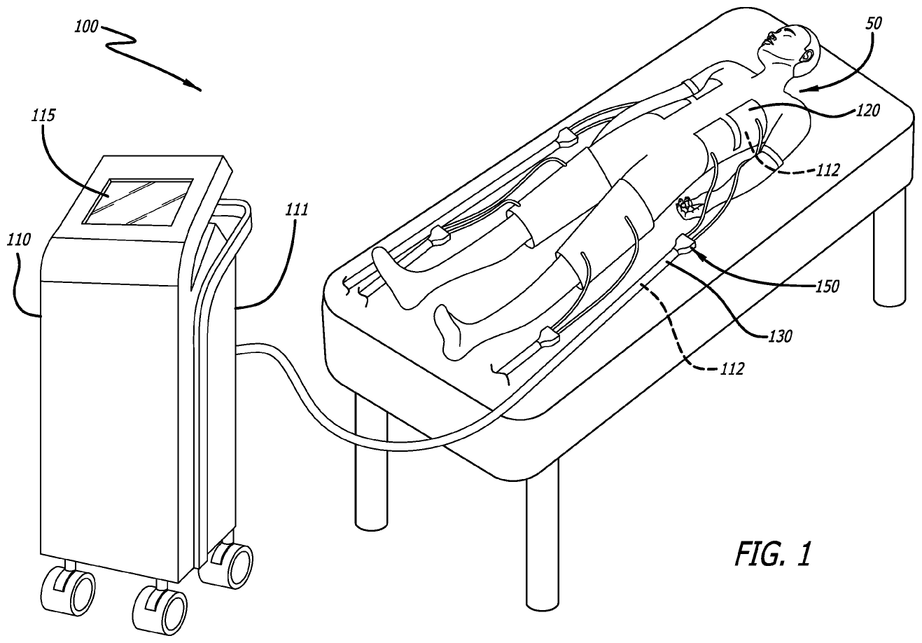

FIG. 1 illustrates a targeted temperature management (TTM) system 100 connected to a patient 50 for administering targeted temperature management therapy to the patient 50 which may include a cooling and/or warming of the patient 50, in accordance with some embodiments. The TTM system 100 comprises a TTM module 110 including a graphical user interface (GUI) 115 enclosed within a module housing 111. The TTM system 100 includes a fluid deliver line (FDL) 130 extending from the TTM module 110 to a thermal contact pad (pad) 120 to provide for flow of TTM fluid 112 between the TTM module 110 and the pad 120. The FDL includes two conduits to facilitate delivery flow of TTM fluid 112 from the TTM module 110 to the pad 120 and return flow TTM fluid 112 from the pad 120 to the TTM module 110. In some embodiments, the two conduits may be attached to each other along a portion of a length of the FDL.

The TTM system 100 may include 1, 2, 3, 4 or more pads 120 and the TTM system 100 may include 1, 2, 3, 4 or more fluid delivery lines 130. In use, the TTM module 110 prepares the TTM fluid 112 for delivery to the pad 120 by heating or cooling the TTM fluid 112 to a defined temperature in accordance with a prescribed TTM therapy. The TTM module 110 circulates the TTM fluid 112 along a TTM fluid flow path including within the pad 120 to facilitate thermal energy exchange with the patient 50. During the TTM therapy, the TTM module 110 may continually control the temperature of the TTM fluid 112 toward a target TTM temperature.

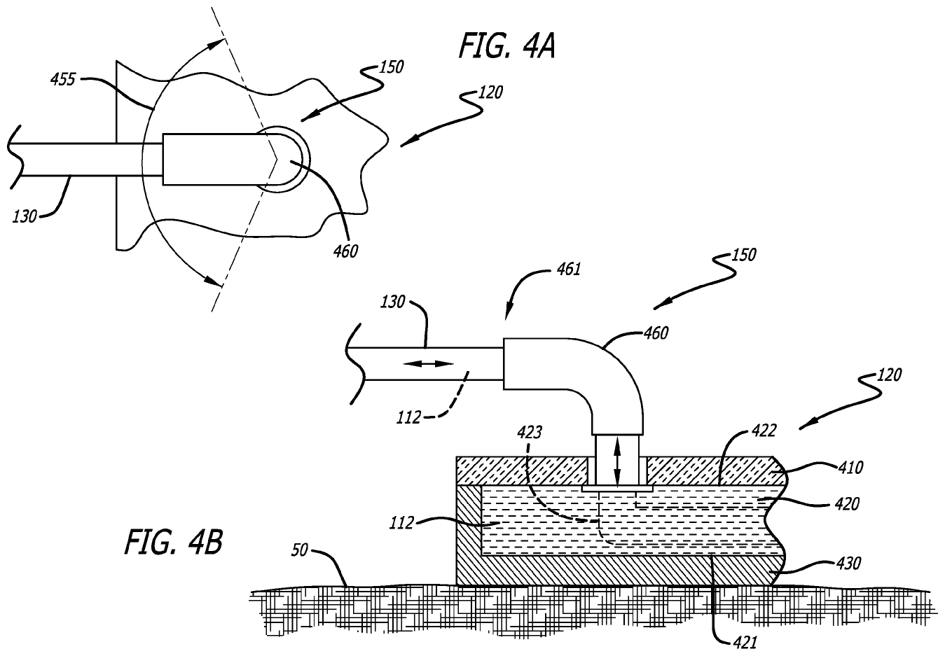

The TTM system 100 may include a connector system 150 to couple the FDL 130 to the pad 120. In some embodiments, the connector system 150 may couple a single fluid conduit of the FDL to the pad 120. Hence, the connection between the FDL 130 and the pad 120 may comprise more than one connector system 150 to couple more than one fluid conduit to the pad 120. The connector system 150 is further described below in FIGS. 4A and 4B.

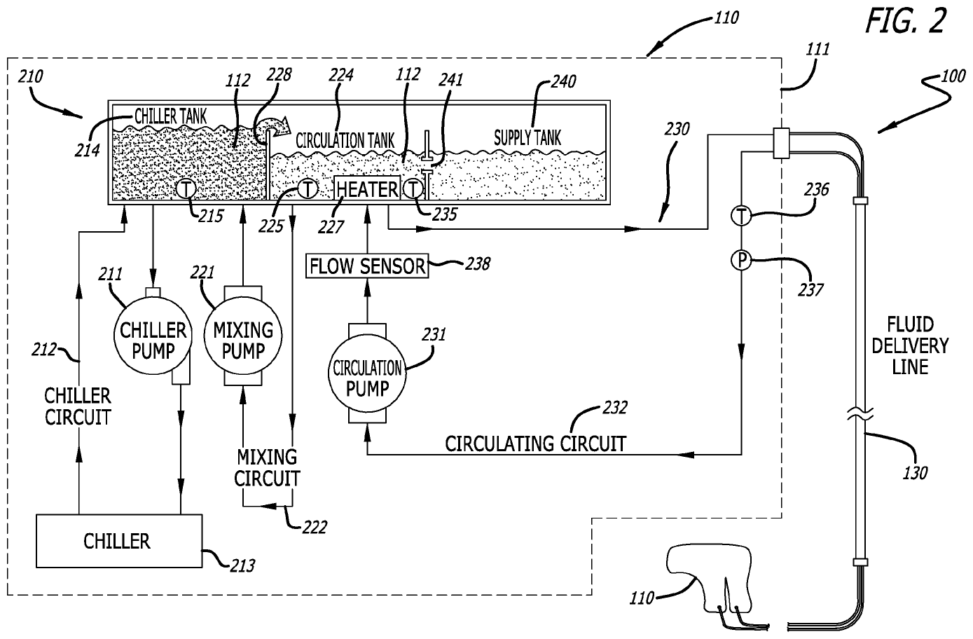

FIG. 2 illustrates a hydraulic schematic of the TTM system 100. The FDL 130 and the pad 120 are disposed external to the housing 111 of the TTM module 110. The TTM module includes various fluid sensors and fluid control devices to prepare and circulate the TTM fluid 112. The fluid subsystems of the TTM module may include a temperature control subsystem 210 and a circulation subsystem 230.

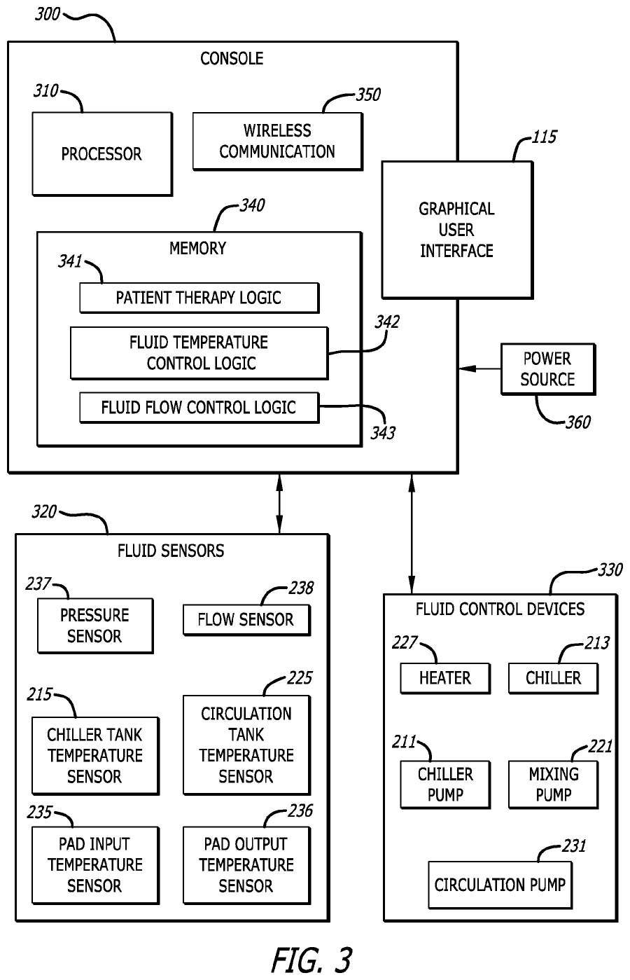

The temperature control subsystem 210 may include a chiller pump 211 to pump (recirculate) TTM fluid 112 through a chiller circuit 212 that includes a chiller 213 and a chiller tank 214. A temperature sensor 215 within the chiller tank 214 is configured to measure a temperature of the TTM fluid 112 within the chiller tank 214. The chiller 213 may be controlled by a temperature control logic (see FIG. 3) as further described below to establish a desired temperature of the TTM fluid 112 within chiller tank 214. In some instances, the temperature of the TTM fluid 112 within the chiller tank 214 may be less than the target temperature for the TTM therapy.

The temperature control subsystem 210 may further include a mixing pump 221 to pump TTM fluid 112 through a mixing circuit 222 that includes the chiller tank 214, a circulation tank 224, and a dam 228 disposed between the chiller tank 214 and circulation tank 224. The TTM fluid 112, when pumped by the mixing pump 221, enters the chiller tank 214 and mixes with the TTM fluid 112 within the chiller tank 214. The mixed TTM fluid 112 within the chiller tank 214 flows over the dam 228 and into the circulation tank 224. In other words, the mixing circuit 222 mixes the TTM fluid 112 within chiller tank 214 with the TTM fluid 112 within circulation tank 224 to cool the TTM fluid 112 within the circulation tank 224. A temperature sensor 225 within the circulation tank 224 measures the temperature of the TTM fluid 112 within the circulation tank 224. The temperature control logic may control the mixing pump 221 in accordance with temperature data from the temperature sensor 225 within the circulation tank 224.

The circulation tank 224 includes a heater 227 to increase to the temperature of the TTM fluid 112 within the circulation tank 224, and the heater 227 may be controlled by the temperature control logic. In summary, the temperature control logic when executed by the processor (see FIG. 3) may 1) receive temperature data from the temperature sensor 215 within the chiller tank and the temperature sensor 225 within the circulation tank 224 and 2) control the operation of the chiller 213, the chiller pump 211, the heater 227, and mixing pump 222 to establish and maintain the temperature of the TTM fluid 112 within the circulation tank 224 at the target temperature for the TTM therapy.

The circulation subsystem 230 comprises a circulation pump 213 to pull TTM fluid 112 from the circulation tank 224 and through a circulating circuit 232 that includes the fluid delivery line 130 and the pad 120 located upstream of the circulation pump 213. The circulating circuit 232 also includes a pressure sensor 237 to represent a pressure of the TTM fluid 112 within the pad 120. The circulating circuit 232 includes a temperature sensor 235 within the circulation tank 224 to represent the temperature of the TTM fluid 112 entering the pad 120 and a temperature sensor 236 to represent the temperature of the TTM fluid exiting the pad 120. A flow meter 238 is disposed downstream of the circulation pump 213 to measure the flow rate of TTM fluid 112 through the circulating circuit 232 before the TTM fluid 112 re-enters that the circulation tank 224.

In use, the circulation tank 224, which may be vented to atmosphere, is located below (i.e., at a lower elevation than) the pad 120 so that a pressure within the pad 120 is less than atmospheric pressure (i.e., negative) when TTM fluid flow through the circulating circuit 232 is stopped. The pad 120 is also placed upstream of the circulation pump 231 to further establish a negative pressure within the pad 120 when the circulation pump 213 is operating. The fluid flow control logic (see FIG. 3) may control the operation of the circulation pump 213 to establish and maintain a desired negative pressure within the pad 120. A supply tank 240 provides TTM fluid 112 to the circulation tank 224 via a port 241 to maintain a defined volume of TTM fluid 112 within the circulation tank 224.

FIG. 3 illustrates a block diagram depicting various elements of the TTM module 110 of FIG. 1, in accordance with some embodiments. The TTM module 110 includes a console 300 including a processor 310 and memory 340 including non-transitory, computer-readable medium. Logic modules stored in the memory 340 include patient therapy logic 341, fluid temperature control logic 342, and fluid flow control logic 343. The logic modules when executed by the processor 310 define the operations and functionality of the TTM Module 110.

Illustrated in the block diagram of FIG. 3 are fluid sensors 320 as described above in relation to FIG. 2. Each of the fluid sensors 320 are coupled to the console 300 so that data from the fluid sensors 320 may be utilized in the performance of TTM module operations. Fluid control devices 330 are also illustrated in FIG. 3 as coupled to the console 300. As such, logic modules may control the operation of the fluid control devices 330 as further described below.

The patient therapy logic 341 may receive input from the clinician via the GUI 115 to establish operating parameters in accordance with a prescribed TTM therapy. Operating parameters may include a target temperature for the TTM fluid 112 and/or a thermal energy exchange rate which may comprise a time-based target temperature profile. In some embodiments, the fluid temperature control logic 342 may define other fluid temperatures of the TTM fluid 112 within the TTM module 110, such a target temperature for the TTM fluid 112 within the chiller tank 214, for example.

The fluid temperature control logic 342 may perform operations to establish and maintain a temperature of the TTM fluid 112 delivered to the pad 120 in accordance with the predefined target temperature. One temperature control operation may include chilling the TTM fluid 112 within the chiller tank 214. The fluid temperature control logic 342 may utilize temperature data from the chiller tank temperature sensor 215 to control the operation of the chiller 213 to establish and maintain a temperature of the TTM fluid 112 within the chiller tank 214.

Another temperature control operation may include cooling the TTM fluid 112 within the circulation tank 224. The fluid temperature control logic 342 may utilize temperature data from the circulation tank temperature sensor 225 to control the operation of the mixing pump 221 to decrease the temperature of the TTM fluid 112 within the circulation tank 224 by mixing TTM fluid 112 from the chiller tank 214 with TTM fluid 112 within circulation tank 224.

Still another temperature control operation may include warming the TTM fluid 112 within the circulation tank 224. The fluid temperature control logic 342 may utilize temperature data from the circulation tank temperature sensor 225 to control the operation of the heater 227 to increase the temperature of the TTM fluid 112 within the circulation tank 224.

The fluid flow control logic 343 may control the operation of the circulation pump 231. As a thermal energy exchange rate is at least partially defined by the flow rate of the TTM fluid 112 through the pad 120, the fluid flow control logic 343 may, in some embodiments, control the operation of the circulation pump 231 in accordance with a defined thermal energy exchange rate for the TTM therapy.

The console 300 may include or be couple do wireless communication module 350 to facilitate wireless communication with external devices. A power source 360 provides electrical power to the console 300.

FIG. 4A shows a top view of a portion of the thermal contact pad 120 including the connector system 150 and the FDL 130 extending away from the connector system 150, in accordance with some embodiments. As illustrated, the connector system 150 may provide for a rotatable connection between the FDL 130 and the pad 120. The rotatable connection may provide for the FDL 130 to swivel through an angle 455 ranging up to about 90 degrees, 180 degrees, or 360 degrees.

FIG. 4B shows a cross-sectional side view of an inlet or an outlet of the thermal contact pad 120 of FIG. 1 in contact with the patient 50, in accordance with some embodiments. The pad 120 may comprise multiple layers to provide multiple functions of the pad 120. A fluid containing layer 420 is fluidly coupled to the FDL 130 via the connector system 150 to facilitate circulation of the TTM fluid 112 within the fluid containing layer 420. The fluid containing layer 420 having TTM fluid 112 circulating therein defines a heat sink or a heat source for the patient 50 in accordance with a temperature of the TTM fluid 112.

The pad 120 may include a thermal conduction layer 430 disposed between the fluid containing layer 420 and the patient 50. The thermal conduction layer 430 is configured to facilitate thermal energy transfer between the fluid containing layer 420 and the patient 50. The thermal conduction layer 430 may be attached to the thermal conduction layer 430 along a bottom surface 421 of the fluid containing layer 420. The thermal conduction layer 430 may be conformable to provide for intimate contact with the patient 50. In other words, thermal conduction layer 430 may conform to a contour of the patient 50 to inhibit the presence space or air pockets between the thermal conduction layer 430 and the patient 50.

The pad 120 may include an insulation layer 410 disposed on the top side of the fluid containing layer 420. The insulation layer 410 is configured to inhibit thermal energy transfer between the fluid containing layer 420 and the environment. The insulation layer 410 may be attached to the fluid containing layer 420 along a top surface 422 of the fluid containing layer 420. In some embodiments, the insulation layer 410 may comprise one or more openings 411 extending through the insulation layer 410 to provide for coupling of the FDL 130 with the fluid containing layer 420.

The connector system 150 may include an elbow 460 to change the direction of FDL 130 extending away from the connector system 150. As shown, the direction of FDL 130 is shifted from a direction perpendicular to the pad 120 to a direction that is substantially parallel to the pad 120. The elbow 460 also establishes an orientation of a distal portion 461 of the FDL 130 to be substantially parallel to the pad 120 and/or the fluid containing layer 420.

In some embodiments, the opening 411 illustrates an inlet port to which the FDL 130 couples such that the TTM fluid 112 may enter into the fluid containing layer 420 and flow freely in a direction as dictated by the negative pressure within the pad 120 resulting from operation of the circulation pump 213. However, in other embodiments, the fluid containing layer 420 may include one or more internal flow paths (illustrated via dashed lines 423) such that the TTM fluid 112 may flow through the internal flow path(s) in a controlled manner in the as dictated by the negative pressure resulting from operation of the circulation pump 213. In some embodiments, e.g., in which FIG. 4B illustrates an inlet port, the TTM fluid 112 exits the pad 120, and specifically the fluid containing layer 420 via an outlet port (not shown) that may resemble the configuration of the as shown in FIG. 4B.

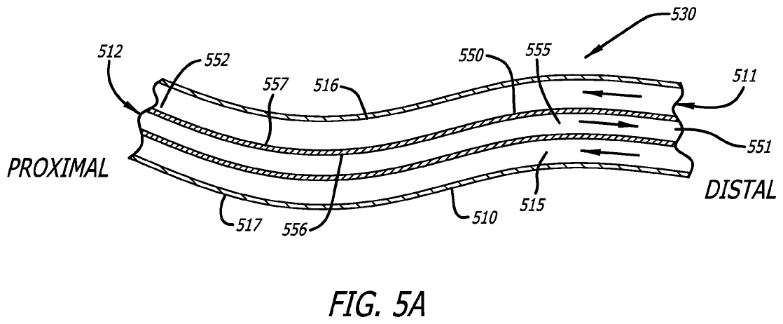

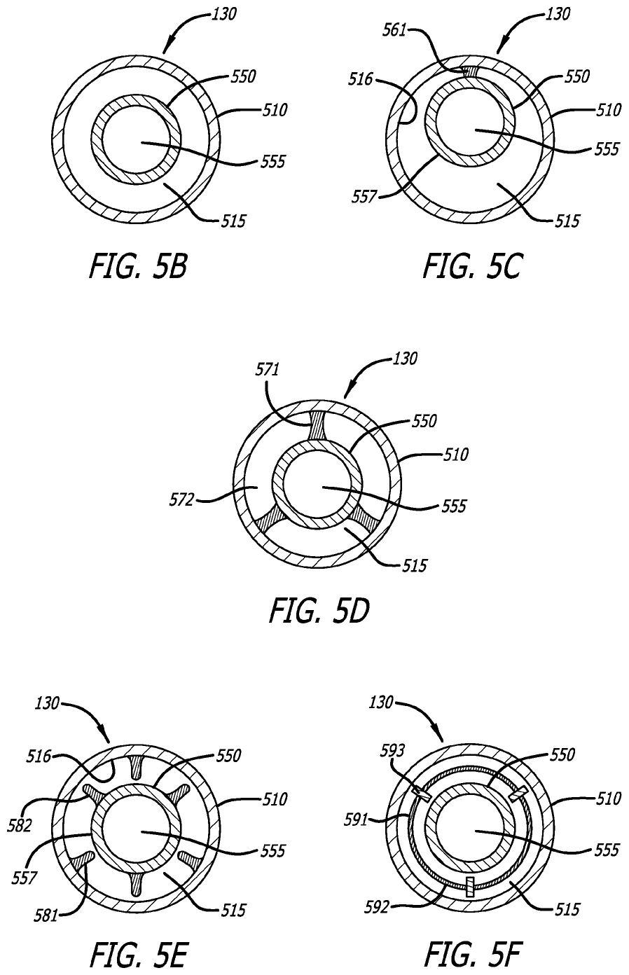

FIG. 5A shows a cross-sectional side view of a conduit set 530. In some embodiments, the FDL 130 may comprise the conduit set 530. The conduit set 530 comprises an outer conduit 510 extending between a distal end 511 and proximal end 512. The conduit set 530 also comprises an inner conduit 550 extending between a distal end 551 and proximal end 552 and defining an inner flow path 555. An outer flow path 515 is defined by the annular area between the inside surface 516 of the outer conduit 510 and the outside surface 557 of the inner conduit 550. The inner conduit 510 and the outer conduit 550 may have the same length. In some embodiments, one of the inner conduit 510 or the outer conduit 550 may be longer than the other.

The outer conduit 510 may be formed of a plastic material via an extrusion process. The outer conduit 510 comprise one or more reinforcements (not shown) extending the length of the outer conduit 510. The reinforcements may inhibit crushing and/or kinking of the outer conduit 510 during use. The reinforcements may also inhibit collapsing of the conduit 510 during use under negative pressure. In some embodiments, the outer conduit 510 may comprise indicia (not shown) on an outer surface 517, such as direction markings, and the outer surface may be smooth or textured.

The outer conduit 510 may be configured to minimize heat transfer between the TTM fluid 112 and the environment. In some embodiments, the structure of the outer conduit 510 may comprise thermal insulative properties, such as a foam structure, for example. To further inhibit heat transfer, the inside surface 516 may be smooth to facilitate fluid flow in the laminar region. Fluid flow in the laminar region may provide for a lower convective heat transfer of the TTM fluid 112 to the inside surface of the outer conduit 510 than fluid flow within a turbulent region. In some embodiments, the inside surface 516 may comprise an antimicrobial coating (not shown) to inhibit bacterial growth.

Similar to the outer conduit 510, the inner conduit 550 may be formed of a plastic material via the extrusion process. The inner conduit 550 may comprise a structure to inhibit collapsing of the conduit 510 during use under negative pressure. The inner conduit 550 may be configured to minimize heat transfer between the TTM fluid 112 flowing within the inner conduit 112 and the TTM fluid 112 flowing along the outside surface 557. In some embodiments, the structure of the inner conduit 550 may comprise thermal insulative properties, such as multiple longitudinal extruded lumens (not shown) within the wall of the inner conduit 550, for example. To further inhibit heat transfer, the inside surface 556 may be smooth to facilitate fluid flow in the laminar region within the inner flow path 555. To further inhibit heat transfer, the outside surface 557 may be smooth to facilitate fluid flow in the laminar region within the outer flow path 515. In some embodiments, the inside surface 556 and the outside surface 557 may comprise an antimicrobial coating (not shown) to inhibit bacterial growth.

FIGS. 5B-5F show cross-sectional end views of different embodiments of the conduit set 530. FIG. 5B shows an embodiment of the conduit set 530 where the inner conduit 550 is not attached to the outer conduit 510. Allowing the inner conduit 550 to float relative to the outer conduit 510 may provide for greater flexibility of the FDL 130.

FIG. 5C shows an embodiment of the FDL where the inner conduit 550 is attached to the outer conduit 510 via longitudinal rib 561. The embodiment of FIG. 5C may comprise a single extrusion. In this embodiment, a longitudinal axis of the inner conduit 550 may be offset from a longitudinal axis of the outer conduit 510. The embodiment of FIG. 5C may comprise more than one rib 561. Offsetting the inner conduit 550 relative to the outer conduit 510 may provide for less heat transfer between the TTM fluid 112 flowing within the inner flow path 555 and the TTM fluid 112 flowing along the outer flow path 515.

FIG. 5D shows an embodiment of the conduit set 530 similar to the embodiment of FIG. 5C. In this embodiment, the inner conduit 550 is concentrically positioned within the outer conduit 510. The inner conduit 550 is attached to the outer conduit 510 via one or more longitudinal ribs 571. The embodiment of FIG. 5D may comprise a single extrusion. In the illustrated embodiment, the outer flow path 515 is divided into three flow paths 572. The number of longitudinal ribs 571 may be 1, 2, 3, 4 or more.

FIG. 5E shows an embodiment where (1) the outer conduit 510 comprises one or more ribs 581 protruding inward from the inside surface 516 and/or (2) the inner conduit 550 comprises one or more ribs 582 protruding outward from the outside surface 557. The ribs 581, 582 may be configured to constrain the inner conduit 550 to be concentric with the outer conduit 510. In this embodiment, inner conduit 550 and the outer conduit 510 may comprise separate extrusions.

FIG. 5F shows an embodiment similar to the embodiment of FIG. 5B in that each of the inner conduit 550 and the outer conduit 510 are separately extruded tubes. In this embodiment a separating structure 591 is disposed between the inner conduit 550 and the outer conduit 510 to constrain the inner conduit 550 to be concentric with the outer conduit 510. The separating structure 591 may comprise a hoop portion 592 coupled to multiple longitudinal rib portions 593. The separating structure 591 may be formed of an extrusion.

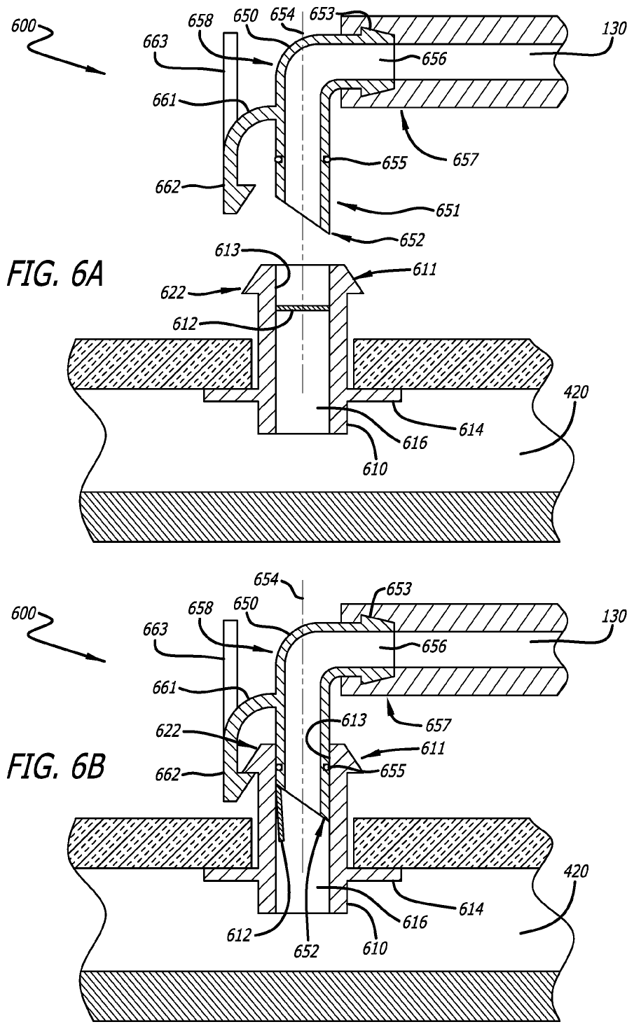

FIG. 6A shows an exploded cross-sectional side view of a connector set 600 including a pad connector 610 and a complementary fluid line connector 650. FIG. 6B shows an assembled cross-sectional side view of the connector set 600. In some embodiments, the connector system 150 may include a connector set such as the exemplary connector set 600 illustrated in FIGS. 6A and 6B. The fluid line connector 650 is attached to the FDL 130 and the pad connector 610 is attached to the fluid containing layer 420. The fluid line connector 650 and the pad connector 610 establish fluid communication between the FDL 130 and the fluid containing layer 420. The connectors 610, 650 are configured to sealably couple together to establish fluid communication between a lumen 656 of the fluid line connector 650 and a lumen 616 of the pad connector 610. The fluid line connector 650 comprises a post portion 651 and the pad connector 610 comprises a post receiving portion 611.

The post portion 651 and the post receiving portion 611 are sized and shaped to form a seal between the pad connector 610 and a fluid line connector 650. The fluid line connector 650 may comprise a sealing member 655 (e.g., an O-ring) to the establish the seal. The pad connector 610 comprises an annular sealing surface 613 to sealably engage the sealing member 655. In some embodiments, the pad connector 610 may comprise a sealing member 655 and the fluid line connector 650 may comprise the annular sealing surface 613. In some embodiments, the post portion 651 may be formed to provide a seal directly with the annular sealing surface 613. In such embodiments, the sealing member 655 may be omitted.

The post portion 651 and the post receiving portion 611 are sized to provide a sliding fit between the pad connector 610 and a fluid line connector 650. The sliding fit may provide for insertion of the post portion 651 within the post receiving portion 611 and provide for rotation of the fluid line connector 650 about the axis 654 with respect to the pad connector 610. In other words, the sliding fit provides for a rotatable connection between the pad connector 610 and the fluid line connector 650. The fluid line connector 650 may comprise an elbow 658 of about 90 degrees so that a fluid line coupling portion 657 extends in a substantially perpendicular direction away from the axis 654.

In some embodiments, the pad connector 610 may comprise a septum 612 extending across the lumen 616 and forming a seal across the lumen 616. In some embodiments, the post portion 651 may comprise a spike 652 configured to rupture the septum 612 when the fluid line connector 650 is coupled to the pad connector 610 as shown in FIG. 6B. In some embodiments, the septum 612 and the spike 652 may be omitted.

The pad connector 610 may comprise a flange 614 to facilitate coupling of the pad connector 610 with the fluid containing layer 420 of the pad 120. The flange 614 may be coupled to the fluid continuing layer 420 via radio frequency welding, ultrasonic welding, adhesive bonding or any suitable coupling process. The fluid line coupling portion 657 may comprise a barb 653 for coupling the fluid line connector 650 to the FDL 130. Alternatively, the fluid line coupling portion 657 may comprise a bonding socket or any other suitable feature for coupling the fluid line connector 650 to the FDL 130.

The connectors 650, 610 may be configured to couple together via a snap fit. In an exemplary embodiment, the snap fit may comprise one or multiple hooks 662 of the fluid line connector 650 configured to engage with an annular ridge 622 of pad connector 610. The hook 662 may comprise a flexible portion 661 to allow for deflection of the hook 662 upon engagement with the annular ridge 622. The snap fit may be configured to facilitate coupling of the connectors 650, 610 via longitudinal displacement of FDL connector 650 with respect to the pad connector 610. In some embodiments, the snap fit may comprise a release mechanism such as the lever arm 663. Operation of the lever arm 663 may disengage the hook 662 from the annular ridge 662 allowing separation of the FDL connector 650 from the pad connector 610.

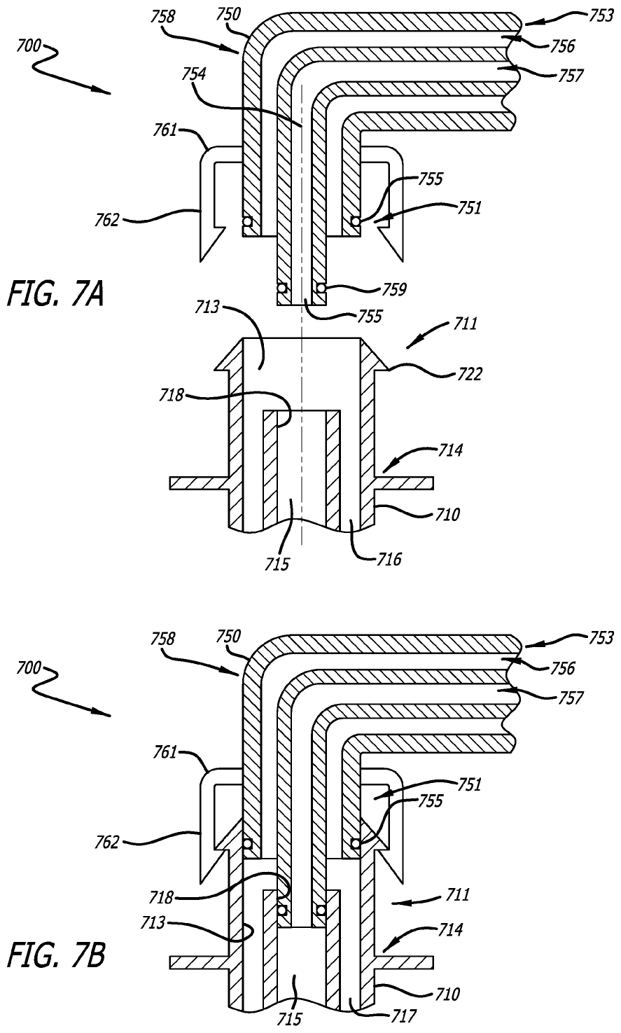

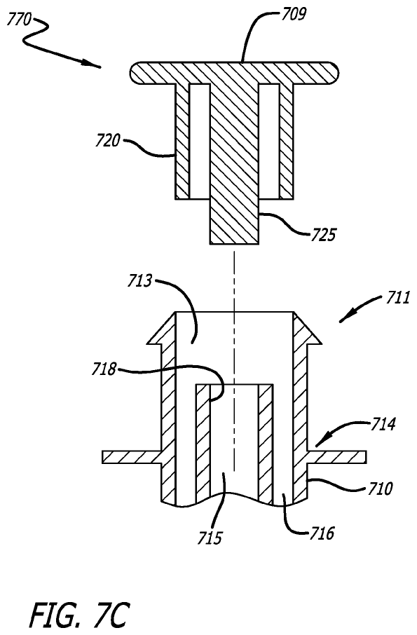

FIG. 7A shows an exploded cross-sectional side view of the connector set 700 including a pad connector 710 and a complementary fluid line connector 750. FIG. 7B shows an assembled cross-sectional side view of the connector set 700. In some embodiments, the connector system 150 may include a multi-luminal connector set such as the exemplary connector set 700 illustrated in FIGS. 7A and 7B. The multi-luminal connector set 700 may correspond with the concentric multi-liminal conduit set 530 described above. The connectors 710, 750 are configured to couple together to establish fluid communication of an inner lumen 755 and an outer lumen 756 of the fluid line connector 750 with an inner lumen 715 and an outer lumen 717, respectively of the pad connector 710.

The fluid line connector 750 comprises a post portion 751 and the pad connector 710 comprises a post receiving portion 711. The post portion 751 and the post receiving portion 711 are sized and shaped to form a seal between the pad connector 710 and a fluid line connector 750. The fluid line connector 750 comprises sealing members 755, 759 (e.g., O-rings) to the establish seals with the annular sealing surfaces 713, 718, respectively of the pad connector 710. In some embodiments, the pad connector 710 may comprise one or both sealing members 755, 759 and the fluid line connector 750 may comprise one or both annular sealing surfaces 713, 718. In some embodiments, the post portion 751 may be formed to provide a seal directly with one or both of the annular sealing surfaces 713, 718. In such embodiments, one or both of the sealing members 755, 759 may be omitted.

The post portion 751 and the post receiving portion 711 are sized to provide a sliding fit between the pad connector 710 and the fluid line connector 750. The sliding fit may provide for insertion of the post portion 751 into the post receiving portion 711 and may provide for rotation of the fluid line connector 750 about the axis 754 with respect to the pad connector 710. In other words, the sliding fit provides for a rotatable connection between the pad connector 710 and a fluid line connector 750. The fluid line connector 750 may comprise an elbow 758 of about 90 degrees so that a fluid line coupling portion 756 extends in a substantially perpendicular direction away from the axis 754.

The connectors 750, 710 may be configured to couple together via a snap fit. The snap fit may be configured to facilitate coupling of the connectors 750, 710 via longitudinal displacement of FDL connector 650 toward the pad connector 610 and thereafter, prevent decoupling when a separating force is applied. In an exemplary embodiment, the snap fit may comprise one or multiple hooks 762 of the fluid line connector 650 configured to engage with an annular ridge 722 of pad connector 710. The hook 762 may comprise a flexible portion 761 to allow for deflection of the hook 762 upon engagement with the annular ridge 722.

The pad connector 710 comprises a pad coupling portion 714 to facilitate coupling of the pad connector 710 with the fluid containing layer 420 (not shown) of the pad 120. The fluid line connector 750 comprises a fluid line coupling portion 753 to facilitate coupling of the fluid line connector 750 to the FDL 130 (not shown).

FIG. 7C shows a front cross-sectional view of a cap 770. In some embodiments, the connector set 700 may include the cap 770. The cap 770 may be applied to the pad connector 710 of the connector set 700. The cap 770 may be configured to provide a seal of the inner lumen 715 and the outer lumen 716 when the cap is attached to the pad connector 710. The exemplary cap 770 may comprise a top wall 709 coupled to circumferential wall 720. The circumferential wall 720 may be sized fit within the receiving portion 711 and provide an inference fit with the annular sealing surface 713. The cap 770 may further comprise a center post 725 sized to fit within the inner lumen 715 and provide an inference fit with the annular sealing surface 718. As such, when the cap 770 is applied to the pad connector, TTM fluid 112 is prevented passing through the outer lumen 716 and the inner lumen 715.

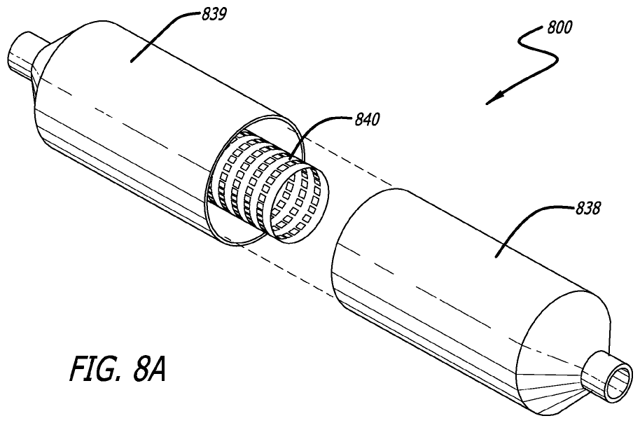

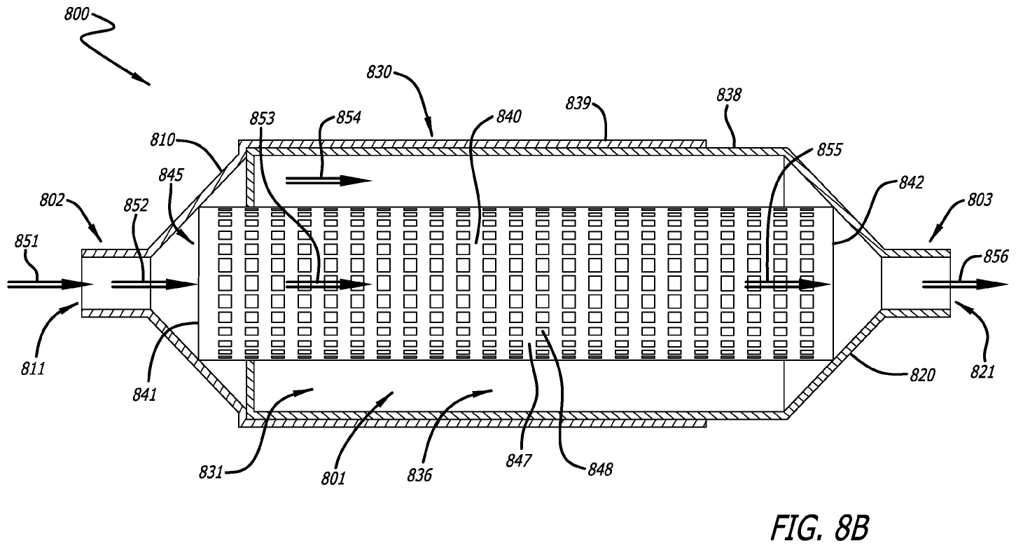

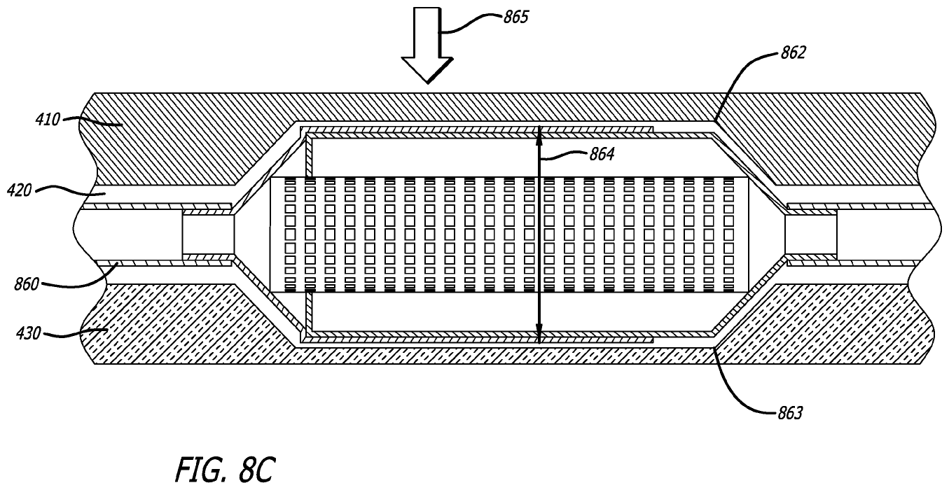

FIGS. 8A and 8B show a filter 800 that may be included with the TTM system 100. The filter 800 may be disposed in line with a TTM fluid flow path of the TTM system 100 so that the circulating TTM fluid 112 flows through the filter 800. The filter 800 may be configured to remove (i.e., filter out) material/particles having a size of 0.2 microns or larger from the TTM fluid 112 without causing a flow restriction of the TTM fluid 112.

The filter 800 comprises a longitudinal shape having a flow path 801 extending from a first end 802 to a second end 803. The filter 800 comprises a diffuser 810 adjacent the first end 802, a nozzle adjacent 820 the second end 803, and a body 830 extending between the diffuser 810 and the nozzle 820. Along the diffuser 810, a cross-sectional flow area of the filter 800 expands from an inlet flow area 811 to a body flow area 831 and along the nozzle 820, the cross-sectional flow area of the filter 800 contracts from the body flow area 831 to an outlet flow area 821. In some embodiments, the inlet flow area 811 and the outlet flow area 821 may be substantially equal.

In some embodiments, the body flow area 831 may be constant along the body 830. In other embodiments, the body flow area 831 may vary along a length of the body 830 such that the body flow area 831 is greater or less along middle portion of the body 830 than at the ends of the body 830. In some embodiments, the body flow area 831 may be circular.

The filter 800 comprises an inner tube 840 disposed within the body 830 extending along the length of body 830. The inner tube 840 may be coupled to the diffuser 810 at a first inner tube end 841 so that TTM fluid 112 entering the filter 800 at the first end 802 also enters the inner tube 840 at the first inner tube end 841. The inner tube 840 may be coupled to the nozzle 820 at a second inner tube end 842 so that TTM fluid 112 exiting the filter 800 at the second end 803 also exits the inner tube 840 at the second inner tube end 842.

The inner tube 840 comprises an inner tube flow area 845 extending the length of the inner tube 840. The inner tube flow area 845 may be greater than the inlet flow area 811 and/or the outlet flow area 821. The inner tube flow area 845 may be constant along the length of the inner tube 840. In some embodiments, the inner tube flow area 845 may vary along the length of the inner tube 840. In some embodiments, the inner tube 840 may comprise a circular cross section. The inner tube 840 and the body 830 may be configured so that the body flow area 831 comprises a combination of the inner tube flow area 845 and an annular flow area 836.

The inner tube 840 comprises a porous a circumferential wall 847. The porous wall 847 may be configured so that TTM fluid 112 may flow through the porous wall 847, i.e., through the pores 848 of the porous wall 847. Consequently, TTM fluid 112 may flow through the porous wall 847 from the inner tube flow area 845 to the annular flow area 836 and from the annular flow area 836 into the inner tube flow area 845.

In use, the longitudinal velocity of the TTM fluid 112 may change along the length of the filter 800. As the volumetric TTM fluid 112 flow through the filter is constant, the longitudinal velocity of the TTM fluid 112 may be at least partially defined by the flow areas of the filter 800 as described below. The TTM fluid 112 may enter the filter 800 at a first longitudinal velocity 851 and decrease along the diffuser so that the TTM fluid 112 enters the inner tube at a second velocity 852 less than the first longitudinal velocity 851. At this point, a portion of the TTM fluid 112 may flow through the porous wall 847 from the inner tube flow area 845 into the annular flow area 836 to divide the fluid flow into a third velocity 853 within the inner tube flow area 845 and a fourth velocity 854 within the annular flow area 836. The fourth velocity 854 may be less than the third velocity 853. A portion of the TTM fluid 112 may then flow back into the inner tube flow area 845 from the annular flow area 836 to define a fifth velocity 855 along the inner tube flow area 845 which may be about equal to the second velocity 852. The TTM fluid 112 may then proceed along the nozzle 820 to define a sixth velocity 856 exiting the filter 800. In some embodiments, the first velocity 851 and the sixth velocity 856 may be about equal.

The filter 800 may be configured to remove harmful bacteria and viruses from the TTM fluid 112 using sedimentation principles. In use, the filter 800 may be oriented horizontally so that the direction of fluid flow through the filter 800 is perpendicular to a gravitational force 865. In some instances, bacteria, viruses, and other particles within the TTM fluid 112 may have a greater density than the TTM fluid 112 and as such may be urged by the gravitational force 865 (i.e., sink) in a direction perpendicular to the fluid flow direction. In some instances, particles within the inner tube flow area 845 may sink toward and through the porous wall 847 into the annular flow area 836. Particles within the annular flow area 836 may then sink toward an inside surface 831 of the body 830 and become trapped adjacent the inside surface 831. The geometry of the filter 800 may be configured to allow 0.2-micron bacteria/virus particles to fall out of the flow of TTM fluid 112 and become trapped along the inside surface 831.

In some embodiments, the filter 800 may be configured so that flow of TTM fluid 112 from the inner tube flow area 845 into the annual flow area 836 my drag particles through the porous wall 847. In some embodiments, the inlet flow area 811, the inner tube flow area 845, and the annual flow area 836 may be sized so that the third velocity 853 is less than about 50 percent, 25 percent, or 10 percent of the first velocity 851 or less. In some embodiments, the body 830 and the inner tube 840 may be configured so that the fourth velocity 854 is less than the third velocity 853. In some embodiments, the fourth velocity 854 may less than about 50 percent, 25 percent, or 10 percent of the third velocity 853 or less.

In some embodiments, the filter 800 may be configured so that the flow within the inner tube flow area 845 is laminar flow, i.e., so that the velocity of the fluid flow adjacent to or in close proximity to an inside surface 841 of the porous wall 847 is less than the velocity at a location spaced away from the inside surface 841. In such an embodiment, the particles may more readily sink toward and through the porous wall 847.

In some embodiments, the filter 800 may be configured so that the fluid flow within the annual flow area 836 is laminar flow, i.e., so that the velocity of the fluid flow adjacent to or in close proximity to inside surface 831 of the body 830 is less than the velocity at a location spaced away from the inside surface 831. In such an embodiment, the particles may more readily sink toward and be trapped along the inside surface 831.

The filter 800 may comprise three components including the inner tube 840 an inner body shell 838, and an outer body shell 839. Each of the three components may be formed via the plastic injection molding process. Assembly of the filter 800 may include capturing the inner tube 840 within the inner body shell 838 and the outer body shell 839 and sliding the inner body shell 838 into the outer body shell 839 wherein the fit between the inner body shell 838 and the outer body shell 839 is an interference fit.

In some embodiments, the filter 800 may be disposed within the pad 120. FIG. 8C shows a detail cross-sectional view of the pad 120 including the filter 800 disposed within the fluid containing layer 420. The filter 800 is coupled in line with an internal flow path 860 within the fluid containing layer 420 so that TTM fluid 12 circulating within the pad 120 passes through the filter 800. The filter 800 may be sized so that the inlet flow area 811 and the outlet flow area 821 are similar to a cross-sectional flow area of the internal flow path 860 within the fluid containing layer 420.

In some embodiments, a thickness of the fluid containing layer 420 may increase adjacent the filter 800 to accommodate a body diameter 864 of the filter 800. To further accommodate the body diameter 864, the insulation layer 410 and/or the thermal conduction layer 430 may comprise internal depressions 862, 863, respectively.

In some embodiments, one or more filters 800 may be disposed in line with the flow of TTM fluid 112 at other locations of the TTM system 100. In some embodiments, one or more filters 800 may be disposed within the TTM module 110. In some embodiments, one or more filters 800 may be disposed in line with the FDL 130. In some embodiments, the filter 800 may be disposed in line with a fluid conduit of the pad external to the fluid containing layer 420 such as a conduit extending between the pad connector 652 and the pad 120.

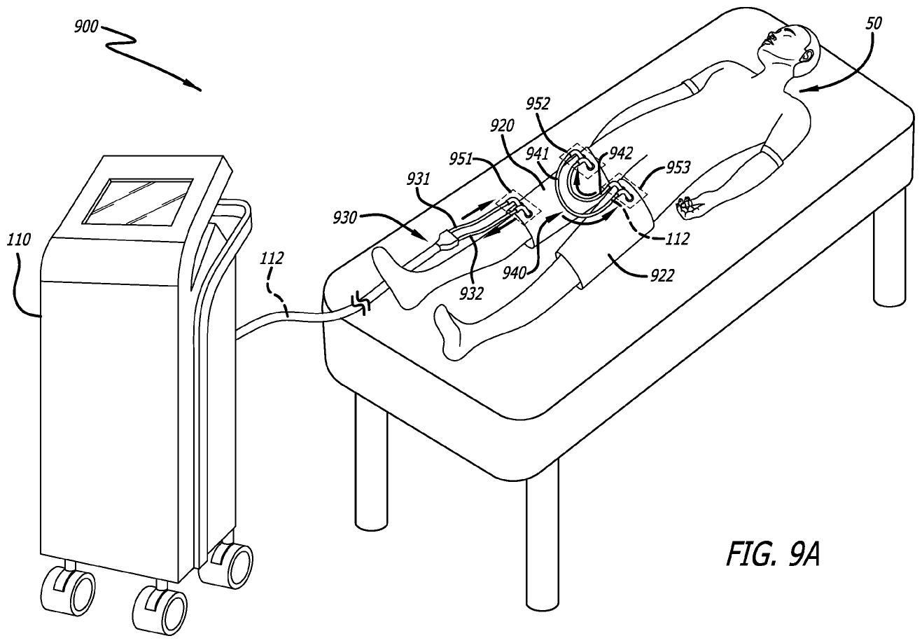

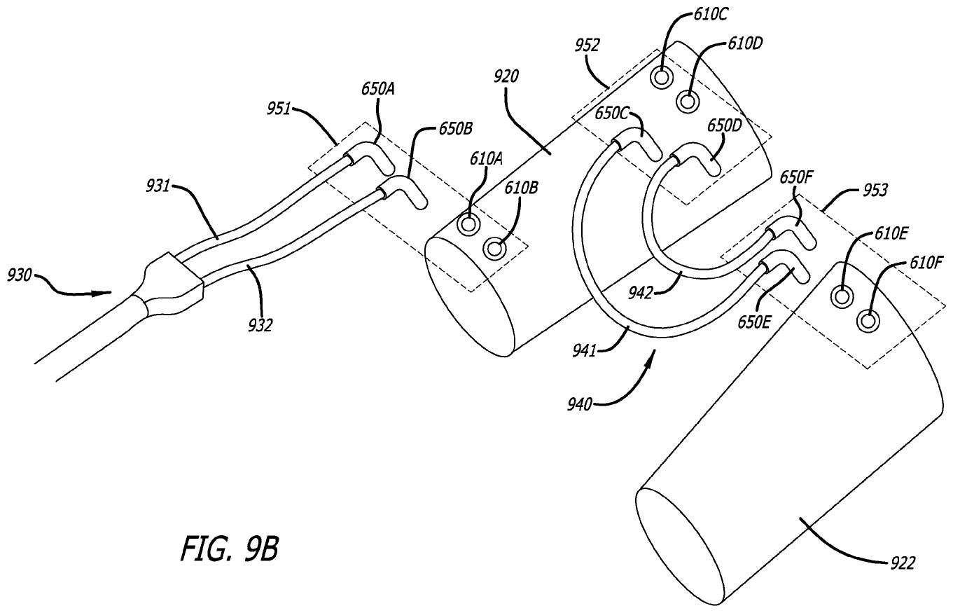

FIG. 9A illustrates the targeted temperature management (TTM) system 900 which in some respects may resemble the TTM system 100 of FIG. 1 in accordance with some embodiments. The TTM system 900 includes a second thermal pad 922 coupled to a first thermal pad 922. FIG. 9B illustrates an exploded assembly view of a portion of the TTM system 900 including the first thermal pad 920 and the second thermal pad 922. In the illustrated embodiment, the first thermal pad 920 is connected to the TTM module 110 via the FDL 930 including a fluid delivery conduit 931 and a fluid return conduit 932. The FDL 930 is connected to the first thermal pad 920 via a first connector system 951. The first thermal pad 920 is further coupled to a fluid transfer line 930 via a second connector system 952 and the fluid transfer line 930 is coupled to the second thermal pad 922 via a third connector system 933. The fluid transfer line includes a fluid delivery conduit 941 and a fluid return conduit 942. In use, TTM fluid 112 flows to the second thermal pad 922 via the fluid delivery conduit 931 of the FDL 930, the first thermal pad 920 and the fluid delivery conduit 941 of the fluid transfer line 940. The TTM fluid 112 flows back to the TTM module 110 via the fluid return line 942 of the fluid transfer line 940, the first thermal pad 920 and the fluid return line 932 of the FDL 930.

Referring to FIG. 9B, each of the connector systems 951, 952, and 953 comprise a pair of connector sets 600 as described with reference to FIG. 6A. As illustrated, the FDL is coupled to the first thermal pad by connecting FDL connector 650A with pad connector 610A and further by connecting FDL connector 650B with pad connector 610B. The first thermal pad 920 is coupled to the fluid transfer line 940 by connecting FDL connector 650C with pad connector 610C and further by connecting FDL connector 650D with pad connector 610D. To complete the TTM fluid circuit, the fluid transfer line 940 is coupled to the second thermal pad 922 by connecting FDL connector 650E with pad connector 610E and further by connecting FDL connector 650F with pad connector 610F. In some embodiments, any or all of the connector systems 951, 952, and 953 may comprise a connector set 700.

Each of the connector systems may be rotatable so that each pad may swivel with relative to each FDL connected thereto. More specifically, the thermal pad 920 may swivel relative to the FDL 930 and/or relative to the fluid transfer line 940. Similarly, the second thermal pad 922 may swivel relative to the fluid transfer line 940. The swivel-ability of the connections systems 951, 952, and 953 may provide a wide variety of placement orientations of the thermal pad 920 and second thermal pad 922 on the patient 50.

In some embodiments, the second thermal pad 922 and the fluid transfer line 940 may be omitted. In which embodiment, the thermal pad 920 may be coupled to the 930 via the connector system 951. The thermal pad 920 may further include the pad connectors 610C, 610D. In some instances, the pad connectors 610C, 610D may each include a septum 612 (see FIG. 6A). In some embodiments, a fluid conduit, such as the fluid conduit 941, may be coupled to the first thermal pad across the pad connectors 610C, 610D.

In some embodiments, any or all of the connector systems 951, 952, and 953 may comprise a connector set 700 (see FIGS. 7A-7B). More specifically, each pad connector may be a pad connector 710 and each fluid line connector may be an FDL connector 750. In such an embodiment, the FDL 930 and/or the fluid transfer line 940 may include the conduit set 530 (see FIG. 5A). In an embodiment where the second thermal pad 922 is omitted, a cap 770 may be applied to each pad connector 710 that is not connected to an FDL connector 750. The cap 770 provides a seal of the pad connector 710 to contain TTM fluid 112 within the fluid containing layer 420 of the pad 120 (see FIG. 4B).

In use, a clinician may apply the first thermal pad 920 and the second thermal pad 922 to the patient 50. During positioning of the first thermal pad 920, the clinician may swivel the first thermal pad 920 with respect to the FDL 930. Similarly, positioning the second thermal pad 922 may include rotating the fluid transfer line 940 with respect to the first thermal pad 920 and/or include rotating the second thermal pad 922 with respect to the fluid transfer line 940.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A targeted temperature management (TTM) system, comprising:
a TTM module configured to provide a TTM fluid;
a thermal pad configured to receive the TTM fluid from the TTM module to facilitate thermal energy transfer between the TTM fluid and a patient; and
a fluid delivery line (FDL) extending between the TTM module and the thermal pad, the FDL configured to provide TTM fluid flow between the TTM module and the thermal pad, wherein:
the thermal pad comprises a pad connector coupled to a corresponding FDL connector of the FDL, and
the FDL connector is rotatable with respect to the pad connector.

2. The TTM system of claim 1, wherein the FDL connector comprises an elbow to facilitate parallel orientation of a distal portion of the FDL with respect to the pad.

3. The TTM system of claim 1, wherein the FDL is multi-luminal.

4. The TTM system of claim 1, wherein the FDL connector is configured to couple to the pad connector via a snap fit.

5. The TTM system of claim 1, wherein at least one of the FDL connector or the pad connector comprises a sealing member and wherein the other of the FDL connector or the pad connector comprises an annular sealing surface, the sealing member and the annular sealing surface configured to facilitate a fluid seal between the FDL connector and the pad connector.

6. The TTM system of claim 1, further comprising a filter disposed in line with a TTM fluid flow path of the TTM system.

7. The TTM system of claim 6, wherein the filter comprises a porous wall disposed parallel to a flow direction of TTM fluid along the TTM fluid flow path.

8. The TTM system of claim 6, wherein the filter is attached to the thermal pad.

9. The TTM system of claim 6, wherein the filter is disposed within a fluid containing layer of the thermal pad.

10. The TTM system of claim 1, further comprising a second thermal pad and a second FDL extending between the thermal pad and the second thermal pad, the second FDL configured to provide TTM fluid flow between thermal pad and the second thermal pad.

11. The TTM system of claim 10, wherein the thermal pad comprises a second pad connector coupled to a corresponding second FDL connector of the second FDL, and wherein the second FDL connector is rotatable with respect to the second pad connector.

12. The TTM system of claim 11, wherein the second thermal pad comprises a third pad connector coupled to a corresponding third FDL connector of the second FDL, and wherein the third FDL connector is rotatable with respect to the third pad connector.

13. A thermal pad for facilitating thermal energy transfer between a TTM fluid and a patient, the pad comprising:
a fluid line configured to transport a TTM fluid between a TTM fluid source and the thermal pad; and
a fluid containing layer configured to contain the TTM fluid and to circulate the TTM fluid within the fluid containing layer to facilitate thermal energy transfer between the TTM fluid and the patient, wherein:
the fluid line is coupled to the fluid containing layer such that a distal portion of the fluid line is oriented parallel to the pad, and
the fluid line is rotatable with respect to the fluid containing layer.

14. The thermal pad of claim 13, wherein the fluid containing layer comprises a TTM fluid flow path, and wherein the pad further comprises a filter disposed in line with the TTM fluid flow path.

15. A thermal pad for facilitating thermal energy transfer between a targeted temperature management (TTM) fluid and a patient, the pad comprising:
a fluid containing layer configured to:
receive a TTM fluid from a TTM fluid source via a fluid line extending between the fluid source and the thermal pad,
contain the TTM fluid, and
circulate the TTM fluid within the fluid containing layer to facilitate thermal energy transfer between the TTM fluid and the patient; and
a connector attached to the fluid containing layer, the connector defining a fluid port of the fluid containing layer, wherein:
the connector is configured to couple to a corresponding fluid line connector to establish fluid communication between the fluid containing layer and the fluid line via a lumen of the connector, and
the connector is configured to swivel with respect to the corresponding fluid line connector.

16. The thermal pad of claim 15, wherein the connector comprises:
a first lumen for transporting TTM fluid to the fluid containing layer; and
a second lumen for transporting TTM fluid away from fluid containing layer.

17. The thermal pad of claim 15, wherein the connector is configured to couple to the corresponding fluid line connector via a snap fit.

18. The thermal pad of claim 15, wherein the connector comprises an annular sealing surface configured to facilitate a fluid seal between the connector and the corresponding fluid line connector.

19. The thermal pad of claim 18, wherein the connector comprises a second annular sealing surface configured to facilitate a second fluid seal between the connector and the corresponding fluid line connector.

20. The thermal pad of claim 15, further comprising a second connector attached to the fluid containing layer, the second connector defining a second fluid port of the fluid containing layer, wherein the second connector is configured to:
couple to a second corresponding fluid line connector of a second fluid line to establish fluid communication between the fluid containing layer and the second fluid line via a lumen of the second connector, and
swivel with respect to the second corresponding fluid line connector when the second connector is coupled to the second corresponding fluid line connector.

21. The thermal pad of claim 20, wherein the second connector comprises a septum extending across the lumen of the second connector to seal the lumen of the second connector, and wherein the septum is configured to be ruptured when the second connector is coupled to the second corresponding fluid line connector.

22. The thermal pad of claim 20, further comprising a third connector attached to the fluid containing layer, the third connector defining a third fluid port of the fluid containing layer, wherein the third connector is configured to:
couple to a third corresponding fluid line connector, and
swivel with respect to the third corresponding fluid line connector when the third connector is coupled to the third corresponding fluid line connector.

23. A method of using a targeted temperature management (TTM) system, comprising:
providing a TTM system comprising:
a TTM module configured to provide a TTM fluid;
a thermal pad configured to receive the TTM fluid from the TTM module to facilitate thermal energy transfer between the TTM fluid and a patient; and
a fluid delivery line (FDL) extending between the TTM module and the thermal pad, the FDL configured to provide TTM fluid flow between the TTM module and the thermal pad;
connecting the FDL with the pad connector via a rotatable connection;
rotating the pad with respect to the FDL from a first orientation of the thermal pad to a second orientation of the thermal pad;
applying the pad to the patient in the second orientation; and
delivering TTM fluid from the TTM module to the thermal pad.

24. The method of claim 23, wherein the TTM system further comprises a second thermal pad and a second FDL, the method further comprising:
coupling the second FDL to the thermal pad via a rotatable connection; and
coupling the second FDL to the second thermal pad via a rotatable connection.

25. The method of claim 24, further comprising rotating the second FDL with respect to the thermal pad from a first orientation of the second FDL to a second orientation of the second FDL.

26. The method of claim 24, further comprising rotating the second thermal pad with respect to the second FDL from a first orientation of the second thermal pad to a second orientation of the second thermal pad.

27. The method of claim 24, further comprising:
applying the second pad to the patient; and
delivering TTM fluid from the thermal pad to the second thermal pad.
